(19) United States
(12) Patent Application Publication
(10) Pub. No.: US 2017/0224612 A1
(43) Pub. Date: Aug. 10, 2017

(54) MUCOADHESIVE CARRIERS OF PARTICLES, METHOD OF PREPARATION AND USES THEREOF

(71) Applicants: VÝZKUMNÝ ÚSTAV VETERINÁRNÍHO LÉKARSTVÍ, Brno (CZ); UNIVERZITA PALACKÉHO V OLOMOUCHI, Olomouc (CZ); TECHNICKÁ UNIVERZITA V LIBERCI, Liberec (CZ); GLOBALACORN LTD., London (GB)

(72) Inventors: Josef MASEK, Brno (CZ); Róbert LUKÁC, Horovce (SK); Milan RASKA, Olomouc (CZ); Pavlína Turánek KNÖTIGOVÁ, Brno (CZ); Jaroslav TURÁNEK, Brno (CZ); Daniela LUBASOVÁ, Jablonec nad Nisou (CZ); Andrew David MILLER, London (GB)

(73) Assignees: VÝZKUMNÝ ÚSTAV VETERINÁRNÍHO LÉKARSTVÍ, Brno (CZ); UNIVERZITA PALACKÉHO V OLOMOUCHI, Olomouc (CZ); TECHNICKÁ UNIVERZITA V LIBERCI, Liberec (CZ); GLOBALACORN LTD., London (GB)

(21) Appl. No.: 15/515,500

(22) PCT Filed: Sep. 29, 2015
(86) PCT No.: PCT/GB2015/052833

§ 371 Date: Mar. 29, 2017

(30) Foreign Application Priority Data
Sep. 29, 2014 (CZ) PV 2014-665

(51) Int. Cl. A61K 9/00; A61K 9/14; A61K 9/19; A61K 9/70

(52) U.S. Cl. CPC A61K 9/006; A61K 9/7007; A61K 9/145; A61K 9/19

(57) ABSTRACT

The present invention relates to a mucoadhesive carrier system, for particles, which comprises nanoscaffold having a nanofibrous layer with a thickness of from 0.1 to 1000 μm, carrying a substance in the form of particles. The mucoadhesive layer, in at least a part of its surface, overlaps the nanoscaffold. A process for its preparation and its use for delivery of the vaccines and therapeutics to mucosal surfaces is also disclosed.

### FIELD OF ART

The present invention relates to mucoadhesive carriers, particularly suitable for carrying and/or administering (active)n substances (such as in the form of particles) to a mucosa of a human or animal.

### BACKGROUND ART

Particulate carriers of vaccines, drugs and other physiologically active substances (e.g., plasmid DNA, siRNA, therapeutic peptides and proteins, antigens, allergens) are used in the treatment and prophylaxis of a number of diseases in humans and animals. Formulations based on nanoparticles and microparticles are usually administered orally and parenterally. The administration of microparticles and nanoparticles to different types of mucous membranes can be non-invasive and painless, with rapid absorption, minimized risk of infection, and bypassing of the digestive system and the portal blood circulation (De Jong W H, Borm P J, Int. J. Nanomedicine. 2008; 3(2): 133-149, Micro- and nanoparticles—medical applications, J\check{a}tariu A, Peptu C, Popa M, Indrei A, Rev. Med. Chir. Soc. Med. Nat. Iasi. 2009 October-December; 113(4): 1160-9).

The total surface area of the oral mucosal lining in a human is approximately 100 cm². The oral mucosa can be divided into the following 3 types: buccal mucosa, sublingual mucosa and palatal mucosa. Individual types of mucosa anatomically can vary in their thickness, degree of the epithelium keratinization, and hence the permeability for drugs, particles and other physiologically active substances. These mucosal categories also differ significantly in the structure (or proportions of the immune cell types). In humans, the sublingual mucosa is the thinnest, without signs of keratinization, whereas buccal mucosa is thicker, but also without signs of keratinization. The palatal mucosa is the thickest one and is keratinized and hence the least permeable for drugs and particles.

In general, the oral mucosa consists of multiple layers, namely a layer of epithelium whose cells flatten towards the surface; basal membrane; lamina propria layer; and submucosal tissue which receives a blood supply and contains numerous nerve endings. The upper layers of the epithelium contain materials of lipophilic nature and intracellular origin stretching between cells, forming a barrier to the passage of particles and substances through the mucosa (Gandhi R B, Robinson J R, Adv. Drug Deliv. Rev., 1994; 13: 43-74).

The main barriers blocking the passage of particles and drugs into, and across, the oral mucosa are (1) the mucin layer on the mucosal surface (Cone R A. Adv. Drug Deliv. Rev., 2009; 61: 75-85), (2) the keratin layer (where present), (3) intercellular lipids of the epithelium (Chen L L, Chetty D J, Chien Y W. Int. J Pharm. 1999; 184: 63-72), (4) basement membrane and (5) an enzymatic barrier (Madhav N V S , Shakya A K, Shakya P, Singh K. J. Control. Release. 2009; 140: 2-11).

Significant external factors influencing the penetration of particles through the mucosa include continuous production of saliva (so washing the mucosal surface and forming a thin film), and movement of the oral mucosa and tongue during speaking, eating, drinking and chewing. Given the similarity of structure and degree of keratinization of mucosa with humans, the pig is currently the most widely used model animal for monitoring the transfer of substances and particles through the oral mucosa (both in-vivo and ex-vivo experiments).

At present it is not clear whether the effectiveness of some particulate carriers of mucosal vaccines fail because of their lack of effect on the immune cells and the immune system, or whether it is caused by insufficient penetration of these particles across the mucosa, especially in model animal species.

Given the barriers and physiological conditions in the oral cavity, the active substances need to be specially prepared and administered by appropriate administration forms. The standard oral drug forms include buccal and sublingual tablets, pastilles, sublingual sprays, oral gels and solutions. However, these drug forms do not allow the ingestion of food or drink, and in the case of sublingual sprays even during speaking. These formulations are preferred for dealing with the administration of low-molecular substances and insulin. More advanced mucoadhesive drug forms can include solutions which form a viscous gel directly on the mucosa, sublingual effervescent tablets and mucoadhesive buccal and sublingual films.

### DISCLOSURE OF THE INVENTION

One aspect of the present invention is to provide an improved mucoadhesive carrier, composition or formulation comprising a nanoscaffold. The nanoscaffold preferably comprises a (nano)fibrous layer or (nano)fibres and may carry or comprise at least one substance (i.e. drug, API or a mixture of substances) preferably comprising, or in the form of, particles. The mucoadhesive layer preferably, over at least part of its surface, overlaps the nanoscaffold. The carrier can be adapted so that (during its use) the nanoscaffold faces the mucosa and/or the mucoadhesive layer serves to (or is capable of) attach or adhere the carrier to the mucosa.

The invention also relates to a mucoadhesive delivery system comprising:

a) a matrix (e.g. a nanoscaffold) comprising at least one active pharmaceutical ingredient (API); and

b) a mucoadhesive (or mucoadhesive means) adapted to adhere, or capable of adhering, the system to a mucosa.

Suitably:

a) the matrix comprises a nanoscaffold (and/or biocompatible polymer(s)), and/or has pores of from 10 nm to 1000 μm;

b) the mucoadhesive is a layer or portion suitably capable of, or adapted to, secure, attach or adhere the system (or matrix or nanoscaffold) to a mucosa (e.g. immune cells, such as dendritic cells and/sub-lingual (cells));

c) the mucoadhesive layer (at least in one part thereof) overlaps or is larger (in surface area) the matrix and/or the system has exposed part of the layer e.g. towards the mucosa; and/or

d) the API (drug, active substance, pharmaceutical, vaccine) is in the form of or comprises particles.

### Mucoadhesive

The mucoadhesive (normally meaning adapted or suitable for adherence, or capable of adhering to, or contact, with a mucosa) can be a layer. At least part of its surface may overlap the nanoscaffold. Thus part of (the surface of) the mucoadhesive (layer) may extend (or overlap) beyond an edge of the nanoscaffold. This (overlapping or exposed) part of the surface of the mucoadhesive layer (e.g. extending beyond the nanoscaffold) can (serve to) attach, or ne capable of attaching, the carrier to the mucosa, such that the nanoscaffold may be adjacent or adhered to the mucosa. The whole structure/system may be thus fixed onto the mucosa by the adherence of part of (the surface of) the mucoadhesive layer, namely extending beyond (or overlapping) the nanoscaffold. In this sense the mucoadhesive can have a larger surface area than the nanoscaffold (or matrix).

### APIs

The substance (e.g. in the form of particles) can comprise the active substance (or API) itself which is suitably carried by the carrier. It can be transported to the target mucosa, e.g. in the form of particles (if the active substance itself is capable of forming particles) or with at least one carrier and/or an excipient, which together may form a particle comprising the active substance (or API). There may be a mixture of active substances (APIs) intended for delivery to the target mucosa, optionally with at least one carrier and/or excipient, which together may form a particle (containing the mixture of active substances).

### Nanoscaffold (or Matrix)

The nanoscaffold (or matrix, the terms are used interchangeably) may be a three-dimensional structure, e.g. a layer. It can be formed by, or comprise, a (layer of) biocompatible polymer(s) or a mixture. Suitably the nanoscaffold comprises one or more nanofibre(s), so thus providing its structure. This may provide space for the API, such as to be absorbed or located or adhered therein. It may also allow the API to leach, or wash out or dissipate or exit from the nanoscaffold, such as over time in a sustained or prolonged release mechanism. The nanoscaffold may thus allow easy entry and/or exit of the API to and therefrom.

These fibre(s) may have a length of from 10, 5 or 1 micron to 0.1, 0.5 or 1 mm. The fibre(s) suitably have a thickness of from 1, 5 or 10 nm to 50, 100, 150, 250 or 500 nm, e.g. from 10 to 150 nm. The nanofiber(s) may thus provide a large (internal) surface area, within the nanoscaffold. Preferably the particle(s) adhere to or are in contact with the nanofiber(s).

It may contain pores with sizes ranging from tens of nanometers to hundreds of micrometers (e.g. 10 nm to 1000 μm, preferably 0.1 to 100 μm). The layer may have a nanofibrous structure, foam structure, or (structure of) plates, crystals or other shapes. It may be from about 15 or 10 to 5 or 3 mm thick (deep), e.g. from 5 or 6 to 8 or 9 mm.

The nanofibrous layer can be a layer of nanofibres with a thickness in the range of 0.1 to 1000 μm, preferably 5 to 50 μm. It can be formed of nanofibres, e.g. comprising biocompatible polymers (or a mixture thereof), preferably with a thickness of 10 or 20 to 2,000 nm, preferably 100 to 800 nm. They may form a net or scaffold, e.g. with a mesh size, that suitably does not (substantially) sterically hinder the movement of the (carried) nanoparticles and/or microparticles therethrough, preferably with a pore size from 0.1 to 100 μm.

Substances suitable for the (production of) nanofibres or present in the nanoscaffold may comprise one or more polyamides, polyurethanes, polyethersulphones, polyvinyl alcohol, polyvinyl butyral, polyacrylonitrile, polyethyleneoxide, polystyrene, polyvinylidene fluoride, polyvinylpyrrolidone, povidone-iodine, alginate, silk fibroin, polyacrylic acid, polyglycolic acid, polyacrylic acid, gelatine, chitosan, collagen, polyaramid, polylactic acid, poly-ε-caprolactone, hyaluronic acid and/or (supersaturated) collagen. The surface of nanofibers can be (further) physically or chemically modified, such as for the purpose of binding and release of particles, in particular macromolecular particles (e.g. proteins, DNA/RNA, polysaccharides) and/or nanoparticles or microparticles of low-molecular substances. Examples of modifications are: change in surface charge and its density, change in surface wettability rate, attachment of ligand(s) for selective binding, such as metallochelating complexes, specific ligands—biotin, monoclonal antibodies and their fragments, peptides, etc.

The fibres may be made by electrospinning. (They may comprise natural and/or synthetic fibre(s)). This may comprise making a solution of the fibres, and the suitably projecting or making them through an (ultrafine) needle.

### Mucoadhesive Component(s)

The mucoadhesive layer usually comprises biocompatible substances or their mixtures. It can have the ability to attach or adhere to mucosal surface, e.g. due to interactions with the mucin layer (present on mucosal surface). The layer may comprise: polyacrylates (carbomers, Carbopol, polycarbophil), cyanoacrylates, tragacanth, xanthan gum, hyaluronic acid, guar gum, gelatine, pectin, polyvinylpyrrolidone, polyethylene oxide, sodium alginate, chitosan, dextran, cellulose derivatives (e.g. hydroxypropylmethylcellulose, hydroxypropylcellulose, hydroxyethylcellulose, hydroxymethylcellulose, sodium carboxymethylcellulose, oxycellulose), poloxamers, copolymers of acrylic and methacrylic acid (Eudragit), lectins, thiolated polymers—thiomers (e.g. chitosan-N-acetylcysteine, chitosan-cysteine, chitosan-thioglycolic acid, carboxymethyl cellulose-cysteine, alginate-cysteine) and the like.

The mucoadhesive layer may further contain one or more plasticizers, e.g. substances providing deformability and/or plasticity of the layer (e.g. glycerol, polyethylene glycol, propylene glycols), substances from the group of phthalates (e.g. dibutyl phthalate), citrates (e.g. triethylcitrate) or surfactants (sodium lauryl sulphate, sodium deoxycholate, sodium cholate, triton and the like).

The mucoadhesive layer and/or the nanoscaffold matrix may also contain one or more excipients facilitating the penetration of particles into the mucosa, preferably substances decreasing the mucus layer viscosity (mucolytics, e.g. acetylcysteine), and/or surface-active substances (sodium deoxycholate, sodium glycocholate, sodium glycodeoxycholate, sodium taurocholate, taurodeoxycholate, sodium cholate, sodium lauryl sulfate, polysorbates (TWEEN80), polyoxyethylene, cetyltrimethylammonium bromide, cetylpyridinium chloride, benzalkonium chloride, etc.) and/or chelating agents (e.g. ethylenediaminetetraacetic acid, EDTA) and/or fatty acids (e.g. oleic acid, capric acid, lauric acid, methyl oleate) and/or polyols (e.g. propylene glycol, polyethylene glycol) and/or dextran sulphate and/or sulfoxides (e.g. dimethyl sulfoxide), and/or Azone® (1-dodecylazacycloheptan-2-one), phosphatidylcholine, lysophosphatidylcholine, methoxysalicylate, menthol, aprotinin, dextran sulphate, cyclodextrins, 23-lauryl ether and the like. The mucoadhesive layer and/or the nanoscaffold may also contain inhibitors of proteolytic enzymes.

### Cover Layer(s)

The carrier/system may further comprise a cover layer. This may be instead of, or in addition to, a mucoadhesive layer. In the former case the cover layer may carry, contain or have the mucoadhesive. Thus the mucoadhesive may be located on a cover layer.

The sequence of layers may be nanoscaffold—mucoadhesive layer (e.g. overlapping the nanoscaffold in at least part of its surface)—cover layer, or mucoadhesive layer may be connected (i.e. in contact with) the cover layer (in part of its surface) and the nanoscaffold can be connected/in contact with the cover layer (over part of its surface).

The cover layer in itself may not have a mucoadhesive (property). It may be inert and/or non-porous or impermeable (e.g. to the API and/or particles). It may comprise a film-forming substance or a substance, which has or can be spun. The substance can be used either alone or in a mixture with other substances mentioned above and/or substances regulating the layer properties (plasticizers, surfactants, agents adjusting pH, ionic strength, etc.).

Examples of suitable substances which can be used to comprise the cover layer are one or more of cellulose derivatives (ethyl cellulose, hydroxypropylmethylcellulose, hydroxypropylcellulose, hydroxyethylcellulose, hydroxymethylcellulose, sodium carboxymethylcellulose, methylcellulose, oxycellulose and cellulose acetate phthalate, celacephate), copolymers of esters of acrylic and methacrylic acids (Eudragit® ), polyacrylates (carbomers, carbopol, polycarbophil), cyanoacrylates, hyaluronic acid, gelatine, pectin, polyvinylpyrrolidone, polyethylene oxide, alginates, gum arabic, shellac, chitosan, waxes, stearic acid, dextran, poloxamers and/or polycaprolactone.

Polyols (glycerol, polyethylene glycol, propylene glycol), substances from the group of phthalates (e.g. dibutyl phthalate) and citrates (e.g. triethyl citrate) can be used as plasticizers. The thickness of the cover layer may be variable, preferably between 0.1 or 1 and 100 or 200 μm. It can be arranged in the form of (or comprise) a polymer film or nanofibers. This layer can block the penetration of particles and molecules (in the direction away from the mucosa) and may ensure a high local concentration (of particles and molecules) to the mucosa, suitably for a sufficiently long period (time interval of the order of tens of minutes to hours). The cover layer can be deposited, for example, by spraying or electrostatic spinning a polymer solution (on the mucoadhesive layer). Suitably the cover layer can prevent the system adhesion to the applicator (or to a finger) during the administration process. It may supply or assist in a required mechanical properties to the entire system: this may ensure easy handling of the formulation and/or after application prevent mucoadhesion to other than the intended site of administration. It may extend the adhesion interval and/or prevent the release of nanoparticles from the nanoscaffold (e.g. into the oral cavity).

The cover layer can be (entirely) insoluble or can gradually dissolve. An (entirely) insoluble layer may extend the interval of the carrier adhesion and/or prevent leakage of particles during the period of presence of the carrier at the site of administration. In a preferred embodiment, the cover layer of the mucoadhesive carrier comprises soluble materials, and these may dissolve at a rate such that dissolution of the individual layers is avoided or reduced before the particles (e.g. API) are released from the carrier. After the release of the particles and subsequent disintegration of the carrier (by an erosion mechanism), and dissolving of different components, it may not be necessary to remove the carrier. In the case of incomplete dissolution (after the required administration interval) fragments may get moved to other parts of the digestive system together with saliva, or with food or drink. This may not be harmful.

### Intermediate Layer

The mucoadhesive carrier or system can preferably comprise an intermediate layer which may be adjacent or in contact with to the nanoscaffold. The intermediate layer may be on the side of the nanoscaffold which is not adjacent to the mucosa. It may be located next to, or between, the mucoadhesive layer and/or matrix/nanoscaffold.

The intermediate layer may comprise a polymer, or another substance, suitably without mucoadhesive properties. Its thickness can be variable, preferably between 0.1 or 1 and 100 or 300 μm. It can be arranged in the form of, or comprise, a polymer (film) or nanofibers. It can be placed or located between the nanoscaffold/matrix and the mucoadhesive layer and/or cover layer (with which it may contact). The intermediate layer can be impermeable, such as to particles e.g. carried or present in the nanoscaffold. This may prevent or reduce their washing out or movement or exit from the carrier e.g. in the direction away from the mucosa. The (insoluble or sparingly soluble) intermediate layer is preferably prepared from or comprise polymeric film-forming substances, commonly used in pharmaceutical technology (or is prepared from or comprise spun polymers arranged into nanofibers).

Examples of suitable materials (for use or formation of the intermediate layer) are one or more of cellulose derivatives (ethyl cellulose, hydroxypropylmethylcellulose, hydroxypropylcellulose, hydroxyethylcellulose, hydroxymethylcellulose, sodium carboxymethylcellulose, methylcellulose, oxycellulose, and cellulose acetate phthalate, celaceát), copolymers of esters of acrylic and methacrylic acids (Eudragit®), polyacrylates (carbomers, Carbopol, polycarbophil), cyanoacrylates, hyaluronic acid, gelatine, pectin, polyvinylpyrrolidone, polyethylene oxide, alginates, gum arabic, shellac, chitosan, waxes, stearic acid, dextran, poloxamers and/or polycaprolactone.

As plasticizers, for example polyols (glycerol, polyethylene glycol, propylene glycol), substances from the group of phthalates (e.g. dibutyl phthalate), citrates (e.g. triethyl citrate) can be used.

The intermediate layer may prevent or reduce particle leakage or movement from nanoscaffold into and/or through the mucoadhesive layer. This leakage may occur due to swelling of the mucoadhesive polymer, e.g. due to osmotic forces and our diffusion of particles.

The individual layers should ideally be prepared in advance and (firmly) attached to, or in contact with each other. They may be deposited in the form of a spray of a solution of a layer-forming substance, e.g. solid particles of a layer-forming substance or a layer-forming substance in the form of nanofibres. The attachment may occur simultaneously with the formation of the layer.

### API and Other Active Substances or Ingredients

The substance (API) may be in the form of particles, which may be incorporated into the nanoscaffold after its formation (they may not be part of nanofibres or part of the nanoscaffold itself). Thus they may be anchored (e.g., by binding or non-covalent interactions) or absorbed. The particles can be liposomes, nanoparticles, microparticles or macromolecules.

Nanoparticles can be particles with a size range from 1 or 10 to 500, 1,000 or 5,000 nm, suitably made from or comprising a biocompatible substance. The most commonly used substances for the preparation of nanoparticles are for example aliphatic polyesters (polylactic acid, polyglycolic acid and copolymers of lactic and glycolic acids, poly-ε-caprolactone), polyalkyl cyanoacrylates, polyhydroxyalkanoates, hydroxymethyl methacrylate, polystyrene sulfonic acid, polystyrene-poly(ethylene glycol), poly(organophosphazene), polyethylene oxide, gelatine and/or polysaccharides (chitosan, hyaluronic acid, alginic acid). Lipids and phospholipids are often used in the formulation of liposomes or lipid-based nanoparticles (LNPs).

These particles may carry (as examples of an API) a drug, antigen, allergen, vaccine, physiologically active substance, nucleic acid, protein, peptide or polysaccharide. The particles may comprise any of the listed agents (e.g. drug, antigen, protein, polysaccharide, nucleic acid), or for example viruses, virus-like particles, LNPs, polymer particles or lipid particles. Suitable particles are in particular: liposomes, polymeric nanoparticles, dendrimers, niosomes, conjugates of low-molecular substances and polymers, complexes of substances with cyclodextrins, nanoemulsions and bacterial envelopes. Furthermore, nanoparticles can be micelles (prepared from surfactants or their mixtures).

### Particles

Microparticles can be particles of a size from 1, 2 or 5 to 10, 20 or 50 μm. They may comprise a biocompatible substance, so suitable for the preparation of microparticles. They may carry or comprise a drug, antigen, allergen, physiologically active substance, nucleic acid, protein, peptide, polysaccharide, or nanoparticles can be formed by any of the above mentioned substances (e.g. drug, antigen, protein, polysaccharide, nucleic acid) or are parts of bacteria, or other pathogens or their fragments. Substances suitable for preparing microparticles are e.g. aliphatic polyesters (polylactic acid, polyglycolic acid and their copolymers, poly-ε-caprolactone), polyalkyl cyanoacrylates, polyhydroxyalkanoates, hydroxymethyl methacrylates, polystyrene sulfonic acid, polystyrene-poly(ethylene glycol), poly(organofosfazen), polyethylene oxide, gelatine and/or polysaccharides (chitosan, hyaluronic acid, alginic acid). Lipids and phospholipids can be used in formulation of liposomes.

Particles may be modified in order to provide them with ability to penetrate the mucin layer without significantly reducing the speed of their diffusion movement (regarding the speed of the diffusion movement of particles in an aqueous medium having a viscosity close to water). This can be achieved by modification of particle surface using polyethylene glycol or another hydrophilic electroneutral polymer, which may impart a surface charge close to zero to particles and their surfaces have a hydrophilic character (Fröhlich E., Roblegg E. J. NanoSci. Nanotechnol. 2014 January; 14 (1): 126-36).

### Other Component(s)

Besides the active substance API, the matrix/nanoscaffold or system may also optionally contain absorption accelerator(s) and/or excipient(s), e.g. an excipient facilitating the release of particles carried to the mucosal surface and/or penetration of the particles through the mucin layer and/or penetration of the particles into the mucosa.

Absorption accelerators (e.g. acetylcysteine), e.g. at the site of administration, may loosen the structure of the adjacent mucin layer and/or loosen the intercellular structure of the epithelium, particularly of the extracellular lipids contained in the upper third of the epithelium.

Excipient(s) may further include, for example, cryoprotectants, antioxidants, stabilizers, antimicrobial agents, surfactants, e.g. detergents, tensids, emulsifiers, mucolytics, sucrose and/or deoxycholate.

Cryoprotective agent(s) may ensure the maintenance of particle stability during the lyophilisation process. Formulation of particles and nanoparticles into a mucoadhesive carrier allows to combine a variety of substances necessary for the functionality and stability of the components during the manufacturing process and the product storage.

The nanoscaffold may serve as a reservoir of microparticles or nanoparticles. These may be reversibly (physically or chemically) adsorbed to nanofibers and/or are (freely) distributed among the fibres. Particles can be spontaneously released from the nanoscaffold e.g. after administration of the mucoadhesive carrier to the mucosa. The nanoscaffold, e.g. serving as a reservoir of particles can have (an appropriate size of) pores in the structure and/or meshes between individual nanofibres, which may not reduce the diffusion movement of the carried particles. The advantage is that the viscosity of the solution inside the nanoscaffold in which the particles move, is not affected by the carrier properties. This problem is encountered in the existing systems which use mucoadhesive gels of high intrinsic viscosity. In order to release the particles from the gel layer, it is, first of all, necessary to hydrate the gel and disintegrate its structure, which reduces the transmission efficiency of the carried particles to the mucosa. The rate of the diffusion movement of particles in the nanoscaffold is only dependent on the viscosity of the outer aqueous environment. Concurrently, the very large surface area of nanofibers or pores is a matrix having a high capacity for adsorption of particles. Simultaneously, a large space is available for depositing the nanoparticles.

The extent and rate of particle release from the mucoadhesive particle carrier may be influenced by both (surface) properties of nanofibres and/or pores in the nanoscaffold and/or surface properties of the (carried) particles. These properties may include the hydrophilic/hydrophobic character of the surface of nanoparticles and nanofibers or pores, surface charge of nanoparticles and nanofibers or pores, shape and size of particles, and structure of the carrying nanofibres or pores. The rate and extent of release of nanoparticles from the nanoscaffold may preferably be increased by surface modification of nanofibers or pores (e.g. by increasing the rate of wettability by surface oxidation of nanofibres or pores in the plasma, by treatment of the nanoscaffold with a sodium hydroxide solution, or by adsorption of suitable surfactants (such as bile salts, sodium lauryl sulphate, and others), and also by surface modification of nanoparticles, for example by influencing the particle charge, or preferably by surface modification of particles with polyethylene glycol. The surface of the particles can be modified by adsorption of a surfactant.

The invention also provides a process for the preparation of a mucoadhesive carriers (or system), wherein a nanoscaffold may be prepared, subsequently attached to or contacted with a mucoadhesive layer and/or to a cover layer. In a preferred embodiment, prior to the attachment, an intermediate layer can be incorporated between the nanoscaffold and the mucoadhesive and/or the cover layer. In one preferred embodiment, the mucoadhesive and/or the cover layer and/or the intermediate layer will be formed, for example, by spraying a polymer solution and drying the solvent. In another preferred embodiment, the mucoadhesive layer and/or the intermediate layer and/or the cover layer will be made in the form of nanofibres (e.g., by electrostatic spinning), and then firmly attached to in the desired order. In another embodiment of the method, nanoscaffold is prepared in situ on the mucoadhesive and/or the cover layer and/or the intermediate layer. If the nanoscaffold is a nanofibrous layer, it can be prepared for example by electrostatic spinning.

A substance, preferably in the form of a solution, colloid, or suspension can be deposited on nanoscaffold, either after its production or after completion of all layers of the mucoadhesive carrier. In a preferred embodiment, the mucoadhesive carrier with the carried substance can be subsequently lyophilized. This enables problem-free long-term storage, important in the case of e.g. vaccines.

### Other Aspects

The invention further provides a (non-invasive) method of administration of a substance (API), e.g. in the form of particles to mucosae, in particular to sublingual, buccal, oral and/or vaginal mucosa. The mucoadhesive carrier, according to this particular invention, can be delivered or contacted directly to the target mucosa either manually or by using a device, e.g. by simply applying or pressing for 1 to 30 seconds, preferably 3 to 10 seconds so that the nanoscaffold is turned towards the mucosa. After releasing the pressure, the carrier can adhere to or attach to the mucosal surface (due to the mucoadhesive forces arising between the mucoadhesive layer and the layer of mucin on the mucosa).

Compared with commonly used methods and carriers (especially the delivery of particles with mucoadhesive properties), this method of non-invasive administration of a carrier can achieve a high local concentration of nanoparticles and microparticles in a close proximity to the mucosal surface for enough time to achieve the required effect of the active substance. These factors can allow a more effective transfer of particles to a mucosa, thus allowing the induction of a therapeutic or prophylactic effect, whilst the administered total dose of particles and substances carried by the particles is lower. The influence of the mucosa and tongue movement to remove particles from the mucosa during common activities such as eating, drinking and speaking can be eliminated or reduced and the effect of dilution of the administered particles with the ingested fluids can be considerably reduced. This can solve or ameliorate the problem of providing uniform dosage of particles and substances carried by the particles, since the mechanisms of particle elimination the mucosal surface can be considerably suppressed. The proposed solution may eliminate the drawbacks of the existing delivery systems to mucosal surfaces.

These systems are based on the delivery of particles with mucoadhesive surface modification, which in turn can adversely affect the penetration of particles to the mucosal surface due to interactions with mucin. Thus, although the particle may remain at the delivery site where the substance can be released, it may be unable to effectively penetrate into the mucosa via the mucin layer. In the prior art, mucin-penetrating particles can be administered, but they (on the contrary) cannot reside in the delivery site for a long period of time because they can be removed by movement of the tongue, and fluids present in the oral cavity, and so carried to other parts of the digestive system. The precondition for dosage uniformity, as well as of other oral dosage forms, is often the limitation of food intake, drinking, or restriction of the tongue movements at a certain period after administration of a dosage form. The use of mucoadhesive gels, which are characterized by high viscosity, can slow down the gel penetration of nanoparticles through the gel to a mucosa.

Preferred features and/or characteristics of one aspect are applicable to another aspect mutatis mutandis.

### BRIEF DESCRIPTION OF DRAWINGS

FIG. 1 illustrates several embodiments of a mucoadhesive carrier in the shape of a round disc according to Example 1.

FIG. 2 shows a diagram of the dissolution rate of the cover layer (Example 1).

FIG. 3 Impregnation of liposomes, surface-modified with polyethylene glycol (PEG liposomes), into the nanofibrous layer prepared from a mixture of polymers chitosan/polyethylene oxide (PEO) (Example 4). A) A transmission electron microscope image shows liposomes adsorbed on the surface of nanofibers. B) An illustrative scanning electron microscopy image shows liposomes adsorbed on the surface of nanofibers. C) Cross-section of a mucoadhesive nanofibre carrier of particles with impregnated liposomes in the nanofibrous layer. D) A detail of the nanofibrous layer penetrated by PEG liposomes (scanning electron microscope image in the frozen state).

FIG. 4 Penetration and adsorption of liposomes with surface-bound model green fluorescent protein (GFP) into the nanofibrous layer prepared from polycaprolactone (PCL). Images of liposomes and nanofibers were taken by confocal microscopy (Example 4). A) Nanofibers labelled using the fluorescent marker lissamine-rhodamine. B) Adsorbed liposomes with surface-bound GFP. C) Overlap of images A and B. D) Detailed view of liposomes with GFP.

FIG. 5 Size and zeta-potential of nanoparticles formed by lactic and glycolic acid copolymer, surface-modified by polyethylene glycol (PLGA-PEG) (Example 4). A) nanoparticle size (Z-diameter 135 nm, polydispersity index: 0.144); B) zeta-potential of PLGA-PEG nanoparticles (−2.21 mV).

FIG. 6 Penetration of the hydrophilic low-molecular weight fluorescent marker 6-carboxyfluorescein and PLGA-PEG labelled nanoparticles into the nanofibrous layer (Example 4). A) Penetration of the fluorescent marker 6-carboxyfluorescein to the nanofibrous layer prepared from a mixture of chitosan/PEO labelled with a fluorescent marker lissamine-rhodamine. B) Penetration of the fluorescent marker 6-carboxyfluorescein into the nanofibrous layer made from PCL. C) Penetration of PLGA-PEG nanoparticles labelled with 3,3′-dioctadecyloxacarbocyanine perchlorate (DiOC18) to the PCL nanofibrous layer.

FIG. 7 Penetration and adsorption of the PLGA-PEG nanoparticles into the nanofibrous layer (Example 4). A) Nanofibrous layer prepared from a mixture of polymers chitosan/PEO. B, C) Adsorbed PLGA-PEG nanoparticles on nanofibers. D) In greater detail. E) PLGA-PEG nanoparticles labelled with the fluorescent label lissamine-rhodamine adsorbed onto nanofibers. F) PLGA-PEG nanoparticles impregnated in the nanofiber layer; particles are placed in the space between nanofibers; they are not adsorbed directly onto the nanofibers.

FIG. 8 The effect of material used, modification of the nanofibre surface and the effect of the presence of surfactants on the amount of released PLGA particles from nanofibers (%) (Example 5).

FIG. 9 The effect of material used, modification of the surface of nanofibres to the amount of the released lissamine-rhodamine liposomes from nanofibres (%) (Example 5).

FIG. 10 Adsorption of microparticles of “bacterial ghosts” (BG) type on a nanofibrous layer made from PCL (Example 6). A) Microparticles of “bacterial ghosts” type impregnated in the nanofibrous layer (scanning electron microscopy, SEM). B) Detailed view of a microparticle adsorbed on the surface of a nanofiber (SEM). C) Fluorescently labelled microparticles of “bacterial ghosts” type impregnated in the nanofibrous layer (confocal microscopy). D) Transverse view; impregnation of particles can be observed along the entire nanofibrous layer.

FIG. 11 Cross-section of porcine sublingual mucosa. Penetration of PEG liposomes into the porcine sublingual mucosa can be observed (Example 7). A) Nuclei B) Fluorescently labelled liposomes, C) Overlap of A) and B), Actin is also labelled.

FIG. 12 Cross-section of a mucoadhesive carrier of particles administered to the mucosa—different layers of the carrier and penetration of PEG-liposomes into porcine buccal mucosa (cryo-SEM) can be observed (Example 7). A) A mucoadhesive nanofiber carrier adhered to the buccal mucosa. B) Detail A), The peeling upper layer is a mucoadhesive layer made from a mixture of polymers of hydroxypropylmethylcellulose (HPMC) and Carbopol 934P, the peeling lower layer is a nanofibrous layer serving as a reservoir for nanoparticles; below the two layers, there is the upper part of the mucosa. C) A detail of the mucoadhesive layer. D) A detail of the nanofiber layer. E) A close contact of the nanofiber layer with the mucous membrane can be seen. F) Detail E), Nanoparticles adhering to nanofibers and particles penetrating through a layer of mucin can be observed.

FIG. 13 Cross-section of porcine buccal mucosa. Penetration of PEG liposomes into porcine buccal mucosa can be observed (Example 7). A) Nuclei B) Fluorescently labelled liposomes, C) Overlap of A) and B), Actin is also labelled.

FIG. 14 A cross-section through porcine sublingual mucosa. Penetration of PLGA-PEG nanoparticles into the porcine sublingual mucosa (formulation containing 1% sodium deoxycholate as accelerator of absorption of nanoparticles) can be seen (Example 7). A) Nuclei, B) fluorescently labelled liposomes, C) Overlap of A) and B), Actin is also labelled.

FIG. 15 Cross-section of porcine sublingual mucosa. The effect of adding 1% sodium deoxycholate on the penetration of PLGA-PEG nanoparticles into sublingual porcine mucosa can be observed (Example 7). A) Penetration of PLGA-PEG nanoparticles from the nanofiber layer to the sublingual mucosa. B) Penetration of PLGA-PEG nanoparticles from the nanofiber layer with the addition of 1% sodium deoxycholate to the sublingual mucosa.

FIG. 16 Nanofiber mucoadhesive carrier of particles used for the experiments on mice (Example 8). A) The entire system with a nanofiber layer in the middle and an overlapping adhesive edge. B) Detail of a nanofibrous layer with adsorbed PLGA-PEG nanoparticles.

FIG. 17. Cross-section of murine sublingual mucosa after in vivo administration of PLGA-PEG nanoparticles (Example 8). A) Specialized immune cells are present in large quantities in the sublingual area. Cells are labelled with the anti-HLA-DR antibody (yellow colour). B) White arrows indicate the PLGA-PEG particles (red) which have been taken up by phagocytic cells (blue colour—cell nuclei). C) A detailed view confirms the internalization of particles within a phagocytic cell.

FIG. 18 The amount of nanoparticles released from the lyophilized nanofibrous layer (Example 9). The effect of 20% sucrose, 1% deoxycholate and a mixture of sucrose and deoxycholate (final concentration 20% and 1%) present in the solution being deposited, on the number of particles released from the nanofibrous layer after lyophilisation.

FIG. 19 Overall view of the mucoadhesive system with a nanofibrous layer for the delivery of nanoparticles (Example 1). A—On the right hand side of the image, an overlapping adhesive margin of the system for nanoparticle delivery can be seen. The nanofibrous layer in the middle serves as a reservoir for nanoparticles. B—Detail of the nanofiber layer deposited on the surface of the mucoadhesive film.

FIG. 20 Cross-section of the mucoadhesive particle carrier with the nanofibrous layer attached to the adhesive layer of the polymer (Example 1). Images were captured using cryo-electron microscopy. The images show a cross-section of the system, which was observed after cracking at −170° C. A) A general view of a transverse crack shows all three layers of the system—cover layer, mucoadhesive layer and nanofibrous layer B) A detail of the cover polymer layer from ethylcellulose on the surface of the mucoadhesive layer (black arrow), C) Detailed view of the nanofiber layer and the mucoadhesive layer adherence, black arrow shows a firm tight adherence, and also shows that neither penetration nor damage of the two layers occurs when the selected method of assembling the layers is used.

FIGS. 21 A and B. Administration of the mucoadhesive carrier of particles to sublingual mucosa in a human (Example 2). The picture was taken 2 hours after the administration; the tongue movements when speaking or ingesting food did not affect the adhesive properties.

FIG. 22 Cross-section of porcine sublingual mucosa with an adjacent mucoadhesive carrier of nanoparticles (Example 3). A), B) General view, the nanofibrous layer can be seen on the surface of porcine sublingual mucosa after 4 h incubation. Remains of the mucoadhesive layer can be seen on the surface of the nanofiber layer. C) A detail of the surface epithelium with the nanofiber layer, a mucin layer can be seen between the nanofiber layer and the epithelial mucosa.

FIG. 23 Penetration of PLGA-PEG nanoparticles into porcine sublingual mucosa and into a regional lymph node after in vivo administration (Example 10). A) PLGA-PEG nanoparticles in the epithelial layer of the sublingual mucosa, A1)—Nuclei A2)—Fluorescently labelled particles, A3)—Overlap. B) PLGA-PEG nanoparticles in the submucosal layer B1)—Nuclei, B2)—Fluorescently labelled particles, B3)—Overlap. C) PLGA-PEG nanoparticles in a regional lymph node, C1)—Nuclei C2)—Fluorescently labelled particles, C3)—Overlap.

### EXAMPLES OF CARRYING OUT THE INVENTION (WHICH ARE NOT LIMITING)

### Example 1

### Preparation of a Mucoadhesive Nanofibrous Carrier of Particles

The mucoadhesive nanofibrous carrier for administration of particles to a mucosal surface consists of several layers. The mucoadhesive layer 2 is a layer that provides adhesion of the whole system to the mucosa and consists of a film of different thickness prepared from substances with mucoadhesive properties or their mixtures. Typically, this layer, from the side intended for the orientation into the oral cavity, is covered with a cover layer 3 which is either slowly soluble, or insoluble in the environment of the oral cavity and has no adhesive properties. It is formed by some film-forming substances used in pharmacy. A film-forming agent is deposited to the mucoadhesive layer as a spray containing the polymer solution and appropriate other substances (e.g. softeners). The nanofibrous layer 1 serves as a reservoir of nanoparticles where the nanoparticles are placed in the space among nanofibres and/or on the surface of nanofibres from where they are released into the mucosa. The nanofibrous layer is deposited to the adhesive layer a) by in situ formation, using the electrostatic spinning process, b) by depositing a preformed nanofiber layer on the mucoadhesive layer.

FIG. 1 illustrates several embodiments of the mucoadhesive carrier in the shape of a round disc. In the first two embodiments, in section A-A or in section B-B, several possibilities of deposition of layers are shown (1—nanofibrous layer 2—mucoadhesive layer, 3—cover layer 4—intermediate layer). The third and fourth embodiments (sections C-C and D-D) show the situation when the nanofiber layer is deposited directly on the cover layer and the mucoadhesive layer is also deposited directly on the cover layer in the parts where the nanofibrous layer is not deposited.

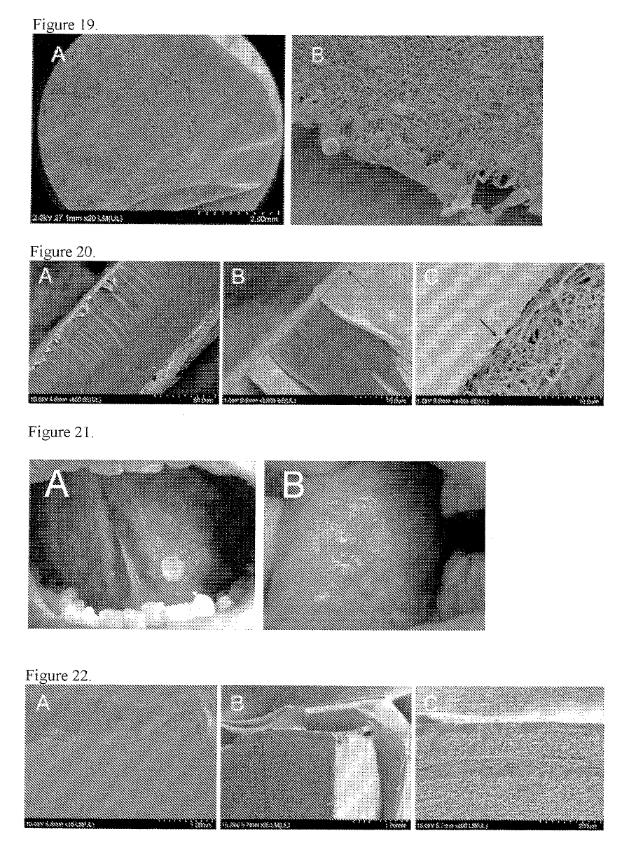

Preparation of the mucoadhesive layer: The layer providing adhesion of the whole system to the target oral mucosa was prepared from a mixture of biocompatible mucoadhesive polymers Carbopol 934P (Noveon, Inc., USA) and Methocel K4M (HPMC) (Colorcon, GB). 300 mg of Carbopol 934P and 100 mg of HPMC were dissolved in 25 ml of water. 20 ml of glycerine, serving as a plasticizer, was added to the polymer solution. The method of evaporating the solvent of the polymer solution at 45° C. was used to produce an adhesive film with suitable mechanical properties. The thickness of the obtained film is approximately 85 μm (see FIG. 20A).

Preparation of the nanofibrous layers: An example is the production of two types of nanofibrous layers:

### Chitosan/Polyethylenoxide (PEO):

The 8% solution of chitosan and the 4% solution of PEO were prepared separately. Chitosan was dissolved in 10% citric acid and PEO was dissolved in distilled water. Both solutions were stirred separately (for 10 hours) using electromagnetic stirrer. In the next operation, sodium chloride at a concentration of 0.85 mol/l was added to the solution of PEO. Subsequently, the polymer solution of chitosan and solution of PEO were combined in order to obtain a solution where the chitosan/PEO weight ratio might be 8:2. The polymer solution was then electrostatically spun partly to a nonwoven material of the spun-bond type (PEGATEX S 30 g/m², anti-static, blue) and partly to the mucoadhesive layer in such a way to form a nanofiber layer in three different square weights, namely 5, 10 and 15 g/m². In order to obtain different surface weights of the nanofibrous layer, it was necessary to electrostatically spin the polymer solution for varying times. The conditions of electrospinning were: the distance of the earthed collector from the electrode 10 cm, voltage 50 kV, temperature 21° C., humidity 60%.

### Polycaprolactone (PCL):

Commercially available PCL was dissolved in a mixture of solvents acetone/ethanol (7/3 v/v) at a concentration of 16%. Electrospinning was carried out under the following conditions: the distance of the earthed collector from the electrode 10 cm, voltage 50 kV, temperature 21° C. Electrospinning was carried out using a nonwoven material of the spun-bond type (PEGATEX S 30 g/m², anti-static, blue). The square weight of the resultant nanofibrous layer was 5 g/m² or 15 g/m². Thickness of the polycaprolactone nanofiber layer having the square weight of 15 g/m² is in the range of 55-70 μm. Thickness of the polycaprolactone nanofiber layer having a square weight of 5 g/m² is in the range of 10-18 μm.

Depositing of the non-adhesive cover layer: The prepared mucoadhesive layer has been on one side coated with a non-adhesive cover layer. The non-adhesive cover layer has improved mechanical properties, which prevented the adhesion of the nanofiber mucoadhesive carriers to other than the target site during the administration. The cover layer should facilitate the administration of the whole system to the target site and handling with it, extend the interval of the residing time of the system on the mucosa and reduce or completely block the diffusion of nanoparticles from the administration site to the space of the oral cavity.

The cover layer may be formed by a polymer soluble in the oral cavity environment or an insoluble polymer. Mechanical properties and the dissolution rate of the carrier are affected by the choice of the cover layer.

Eudragit® 100-55L was chosen as an example of coating having suitable mechanical properties soluble in the oral cavity environment. Eudragit® 100-55L was applied by spraying in the form of a 1% ethanol solution with the addition of propylene glycol as a plasticizer (0.25 g Eudragit® 100-55L, 35 μl of propylene glycol, 25 ml ethanol (96%)). The resulting coating thickness depending on the amount of the applied polymer solution was in the order of several hundred nanometers to μm units (see FIG. 20).

To prepare a cover layer insoluble in the oral cavity environment, ethyl cellulose polymer was used as an example. The polymer was applied as a spray of a 2.5% solution of ethyl cellulose in ethanol (0.25 g ethyl cellulose, 17.5 μ; propylene glycol, and 10 ml ethanol (96%)) on the surface of the mucoadhesive layer (FIG. 20). For faster evaporation of the solvent, the mucoadhesive layer was placed on a heated plate at 50° C. Both ethyl cellulose and Eudragit® 100-55L are commonly used in preparing human pharmaceutical formulations. They are nontoxic and safe.

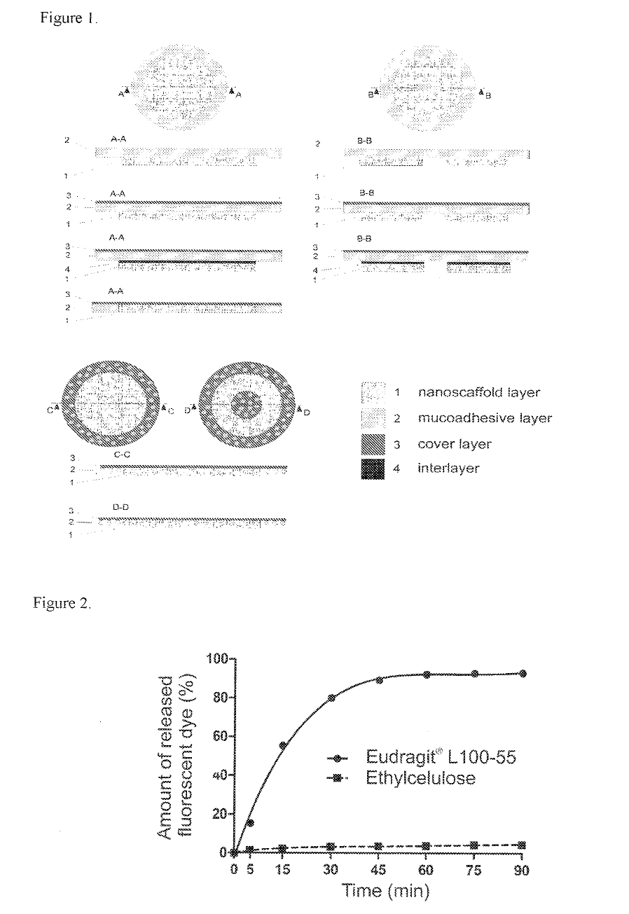

Determination of the dissolution rate of the covering polymeric film: To determine the dissolution rate of the covering polymer film, hydrophilic fluorescent label 6-carboxyfluorescein, used for labelling of the nanofiber mucoadhesive carrier, was added to the polymer solution. The carrier was placed on the bottom of a 100 ml vessel. Phosphate buffer with pH 6.0 was chosen as the dissolution medium. The dissolution rate of the cover layer was determined as the concentration of 6-carboxyfluorescein buffer increasing in time. While the coating prepared from Eudragit 100-55L completely dissolved in approximately 30 minutes, coating prepared from ethyl cellulose remained almost undissolved during the monitoring period (see FIG. 2).

Assembling the nanofiber layer with the mucoadhesive layer: The nanofibrous layer made from a mixture of polymers chitosan/PEO with the thickness of 10 μm was attached by pressing against the mucoadhesive layer (mixture of HPMC and Carbopol 934P in a weight ratio 1:3) after slight moistening of the mucoadhesive layer by water steam. Whereas no penetration of the mucoadhesive layer into the layer of nanofibres occurs, their tight and mechanically durable attachment develops. The elasticity of the two layers will ensure intimate contact with the target tissue.

Preparation of the nanofibrous layer by the electrostatic spinning process onto the mucoadhesive layer: The nanofibrous layer can be prepared by the process of electrostatic spinning of a polymer solution directly on the mucoadhesive layer.

The mucoadhesive layer was placed on a collector, below which a spinning electrode was located. The polymer solution was dispensed to the spinning electrode at the volume of 1.5 ml and spun directly onto the mucoadhesive layer under the following conditions: the distance of the gathering collector from the earthed electrode was 10 cm, voltage 30 kV.

In both examples of the attachment of the layers of the carrier, mechanically durable attachment is achieved, without affecting the structure and function of either layer.

FIG. 19 illustrates a system of the nanofiber layer and the mucoadhesive layer; FIG. 20 shows a cross-section of the nanofiber layer system, nanofiber layer and cover layer.

### Example 2

### Method of the Mucoadhesive Carrier Administration Onto the Mucosa

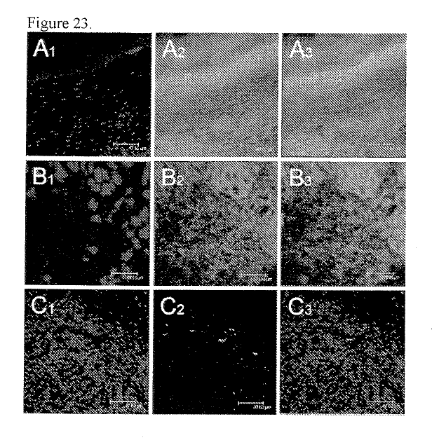

A mucoadhesive nanofiber carrier of particles is administered onto the oral mucosa, particularly sublingual and buccal which is not keratinized in humans. The nanofiber mucoadhesive carrier is placed on a finger with the non-adhesive side against the finger and by a slight pressure is applied to the target site in the oral cavity, for example to the underside of the tongue (sublingual mucosa) or to the buccal mucosa, for approximately 5 seconds, before adhesion is created between the mucoadhesive side of the system and the mucosa. Alternatively, a suitable applicator can be used. The applicator is particularly advantageous in veterinary medicine. It was verified that 3 hours after the administration, the tongue movements during speaking or ingesting food did not affect the adhesive properties of the carrier (FIG. 21).

### Example 3

### Ex-Vivo Administration of the Mucoadhesive Carrier on the Mucosa

Porcine sublingual mucosa is a model of qualities that are very close to humans. After the removal from a freshly killed animal, sublingual mucosa and buccal mucosa were washed with saline and were used immediately for the administration of nanoparticles using a carrier. Firstly, the nanofiber layer of the carrier was saturated with a solution of liposomes or nanoparticles prepared from the mixture of PLGA and PLGA-PEG polymers with a concentration of 20 mg/ml. Further, the carrier with liposomes or nanoparticles was placed on a finger with the non-adhesive side against the finger and exerting a slight pressure for about 5 seconds it was applied to the target site. In order to study the penetration of liposomes and PLGA nanoparticles into the tissue, the system administered to the mucosa was incubated in a moist chamber at 37° C. for 4 hours. Mucosal surface was kept moistened with saline to simulate saliva production. The situation after the 4-hour incubation is shown in FIG. 22.

Preparation of liposomes: Liposomes were prepared by a lipid film hydration method. The final liposome size was achieved by extrusion through polycarbonate filters with pores of a defined size of 100 nm.

Composition of liposomes (fluorescently labelled): 10 mol % 1,2-distearoyl-sn-glycero-3-phosphoethanolamin-N-[amino(polyethylen glycol)-2000] (DSPE-PEG); 89.5 mol % egg phosphatidylcholine (EPC); 0.5 mol % lissamine-Rhodamine.

### Example 4

### Impregnation of the Nanofibrous Layer With Nanoparticles

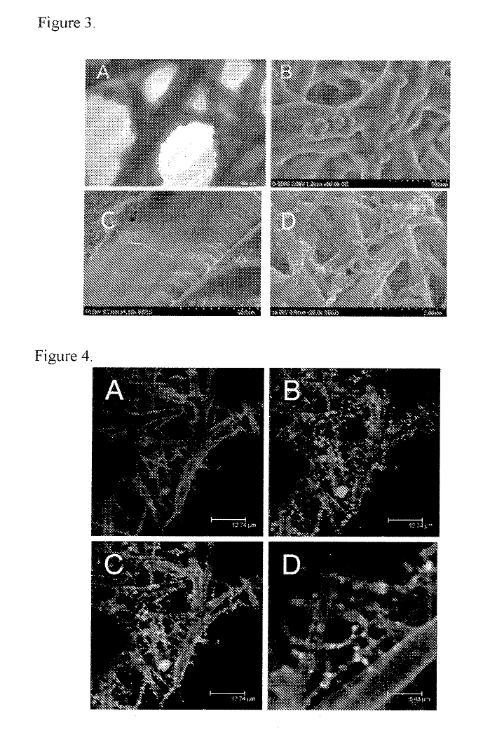

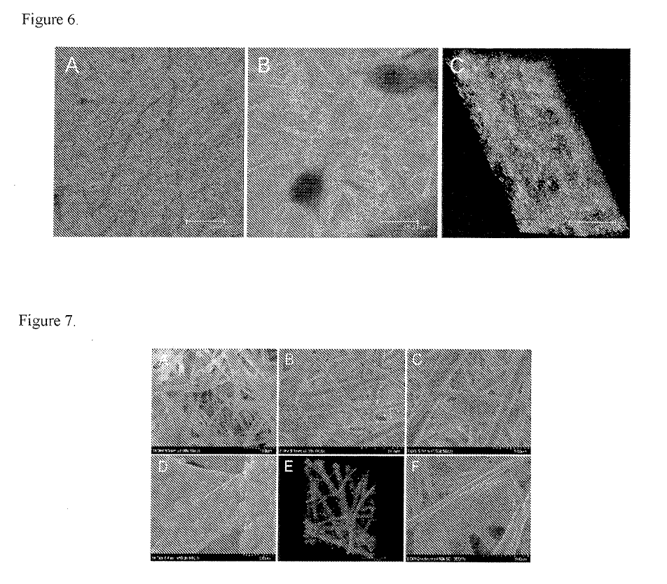

The nanofibrous layer was impregnated with a suspension of nanoparticles (liposomes or PLGA-PEG nanoparticles). Depending on the properties of nanoparticles, the nanofiber layer and on the method used, the nanoparticles were adsorbed onto the nanofibre surface or formed inclusions in the space between the nanofibers (FIGS. 3, 4, 6 and 7). The carrier of nanoparticles prepared in this way is applied immediately after the deposition of nanoparticles. It is also possible to stabilize the particles in the nanofibrous layer for long-term storage. The particles are kept in the nanofibrous layer after the solvent evaporation. However, stabilization of particles by the lyophylisation process with cryoprotectants added into the solution of nanoparticles appears more advantageous. One possibility of applying nanoparticles on the nanofiber layer is the application of nanoparticles in the solution after assembling the nanofiber layer with the adhesive layer. This was carried out by turning down the system with its non-adhesive side after which the solution of nanoparticles was applied onto the surface of the nanofibrous layer. In this way the nanoparticles spontaneously spread evenly and impregnated the nanofibrous layer. For impregnating a nanofiber layer having an area of 0.5 cm² and a thickness of 15 μm, 2 μl of particle suspension was used. The concentration of the nanoparticles (liposomes or PLGA-PEG) was 20 mg/ml.

Another application method is the immersion of the nanofiber layer in the solution of nanoparticles. This was performed by immersion of the nanofibrous layer into a solution of nanoparticles of the required concentration. Where required by the nanofiber properties, a tray-shaped ultrasonic bath was used to facilitate the impregnation of particles. For impregnating a nanofiber layer having an area of 0.5 cm² and a thickness of 15 μm, 100 μl of a solution of nanoparticles (liposomes, liposomes with surface-bound model protein or PLGA-PEG nanoparticles) was used at a concentration of 20 mg/ml. The nanofibrous layer was immersed to this solution for 5 minutes. In the case of penetration of PLGA-PEG nanoparticles, the vial containing the solution of nanoparticles was immersed in a tray-shaped ultrasonic bath to facilitate impregnation.

Facilitation of the impregnation of particles into the nanofibrous layer and influencing the rate of adsorption of nanoparticles onto the surface of nanofibers was be achieved by physical or chemical modification of the surface of nanofibers.

Preparation of liposomes: Liposomes were prepared by a lipid film hydration method. The final liposome size was achieved by extrusion through polycarbonate filters with pores of a defined size of 100 nm. For the preparation of liposomes with surface-bound protein, the prepared liposomes were mixed with the recombinant His-tagged protein GFP in a defined ratio. The protein was bound to the surface of liposomes by means of metallochelation.

Composition of PEG liposomes: 10 mol % DSPE-PEG; 90 mol % EPC.

Composition of liposomes for surface modification by GFP protein: 5 mol % of 1,2-di-(9Z-octadecenoyl)-sn-glycero-3-[(N-(5-amino-1-carboxypentyl) iminodiacetic acid)succinyl] (nickel salt) (DOGS-NTA-Ni); 19 mol % of 1-hexadecanoyl-2-(9Z-octadecenoyl)-sn-glycero-3-phospho-(1′-rac-glycerol) (POPG); 76 mol % EPC.

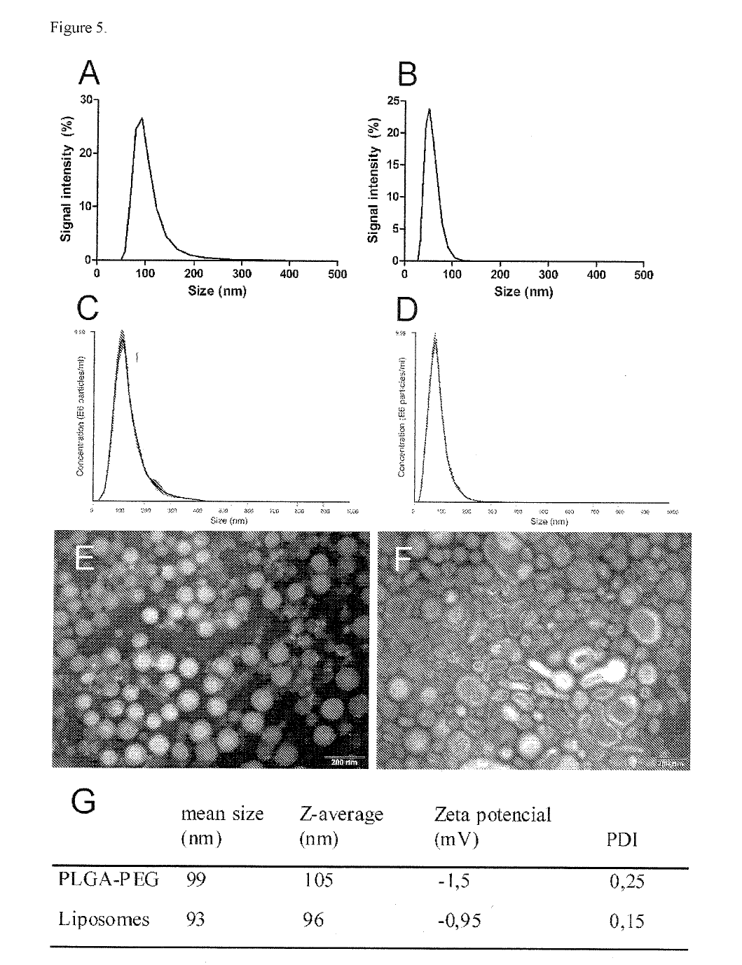

Preparation of PLGA-PEG nanoparticles: Nanoparticles were prepared by dissolving 25 mg of PLGA (lactic acid: glycolic acid (50:50), Mw 30,000 to 60,000) (Sigma-Aldrich) and PLGA-PEG (PEG Mw 5,000, PLGA Mw 55,000) (Sigma-Aldrich) in 1 ml of dichloromethane. 1 ml of the organic phase was emulsified in 5 ml of 0.7% sodium cholate by sonication at 70% amplitude, by 1-second pulses for 5 min. The emulsion obtained in this way was diluted with 20 ml of 0.5% sodium cholate, and the organic phase was removed from the emulsion in an evaporator under reduced pressure. Large aggregated particles were removed by centrifugation at 500 rpm/min. From the resulting nanoparticle suspension, excess cholate was removed by diafiltration (Spectrum). The particles were concentrated to the desired concentration in the same way. The size (A) and zeta potential (B) of PLGA-PEG nanoparticles were measured by the dynamic light scattering method; the result is shown in FIG. 5.

### Example 5

### Release of Nanoparticles From the Nanofibrous Layer—the Effect of the Material Used and the Effect of Surface Modification of Nanofibres on the Release of Nanoparticles

Interactions of nanoparticles and nanofibers in the carrier matrix is affected by the surface properties of nanoparticles and nanofibers. The release rate and releasable amount of nanoparticles can be affected by the polymer used for the production of nanofibres and its subsequent surface modification. Surface properties of nanoparticles can also be modified in order to improve their release from the nanofibrous layer. Nanoparticles must meet a number of criteria so that they might be able to pass through the mucosal barriers and, therefore, it is very advantageous to modify the surface properties of nanofibers by chemical or physical means. Listed below are examples of possible modifications:

1) Chemical treatment of nanofibers made from PCL

2) Physical adsorption of surfactants onto the surface of nanofibers made from PCL

The numbers of PLGA-PEG nanoparticles and PEG liposomes released from the nanofibrous layer penetrated by the given type of nanoparticles were monitored.

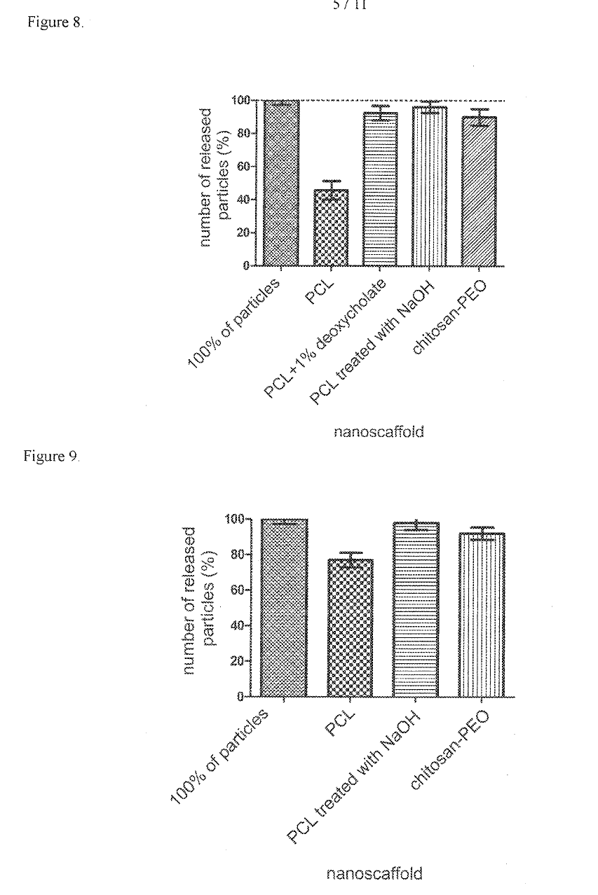

Furthermore, some surfactants (e.g. sodium lauryl sulphate, sodium deoxycholate and others, known as absorption accelerators) increase permeability of the mucosa for drugs and nanoparticles (FIG. 8).

Then, two functions can be fulfilled by the present surfactants. They increase the penetration of nanoparticles applied to the mucous membrane by changing the mucosal barrier functions and enhancing the release of nanoparticles from the nanofiber layer.

Chemical modification of nanofibers: The nanofibers made from PCL are of hydrophobic character. To increase wettability and limit the hydrophobic interactions with nanoparticles, their surface was modified by immersion of the nanofibrous layer in 3 M NaOH for 10 min. The nanofibrous layer was then rinsed with water several times (FIG. 9).

Deposition of the nanoparticles solution of on the nanofiber layer: The nanofibrous layer of PCL was penetrated by its immersion in a solution of PLGA-PEG nanoparticles or PEG liposomes at a concentration of 20 mg/ml for 5 min.

Releasing of nanoparticles from the nanofiber layer to the solution: The nanofibrous PCL layer of a round shape with the surface area of 0.5 cm² penetrated by the solution of PLGA-PEG nanoparticles was immediately placed in 0.5 ml of aqueous solution. The nanofibrous layer was incubated for 30 minutes under gentle shaking. The solution obtained was diluted as necessary to match the parameters for nanoparticle concentration measurement using the chosen method.

Determination of the number of the released nanoparticles: The number and size of released PEG-PLGA nanoparticles were determined by “Nanoparticle tracking analysis” (NanoSight, Malvern, UK). The amount of released liposomes was determined as the solution fluorescence intensity after incubation of the nanofibrous layer measured at excitation of 560 nm and emission of 583 nm. The obtained data was adjusted according to the dilution factor of the measured solution and the amount of the released particles was calculated (%).

Adsorption of surfactants: The effect of adsorption of surfactants onto the surface of nanofibers was studied after penetration of the nanoparticle penetration of the nanofibrous layer. The solution of nanoparticles contained sodium deoxycholate at a concentration of 1%. In a second embodiment, the nanofibrous layer was first immersed in a solution of 1% sodium deoxycholate, rinsed several times with water and dried. Then it was impregnated with a solution of nanoparticles.

### Example 6

### Adsorption of Microparticles of the “Bacterial Ghosts” Type

Microparticles are also be used as vaccine delivery systems. One type of such microparticles are empty bacterial envelopes termed as “bacterial ghosts” (BG).

BG are non-pathogenic particles derived from bacterial cells. They contain the bacterial cell wall, including antigenic structures, against which a specific immune response is induced. The intracellular content is removed for example by osmotic shock, and therefore the particles obtained in this way are unable to further propagate. Due to the natural presence of a number of substances recognized by the immune system known as danger signals, such particles provide a complex signal for inducing a specific immune response against the antigenic structures present. Bacterial ghosts can potentially be used as vaccine particles for mucosal administration.

Fluorescent labelling of “bacterial ghosts”: Bacterial ghosts (prepared from Escherichia coli) were ultrasonically dispersed in water. Fluorescent marker DiOC18 dissolved in ethanol was added to the suspension of bacterial particles, the mixture was further ultrasonicated for 1 min so that the fluorescent marker might incorporate in the wall of the particles. Centrifugation and washing removed the excess fluorescent marker.

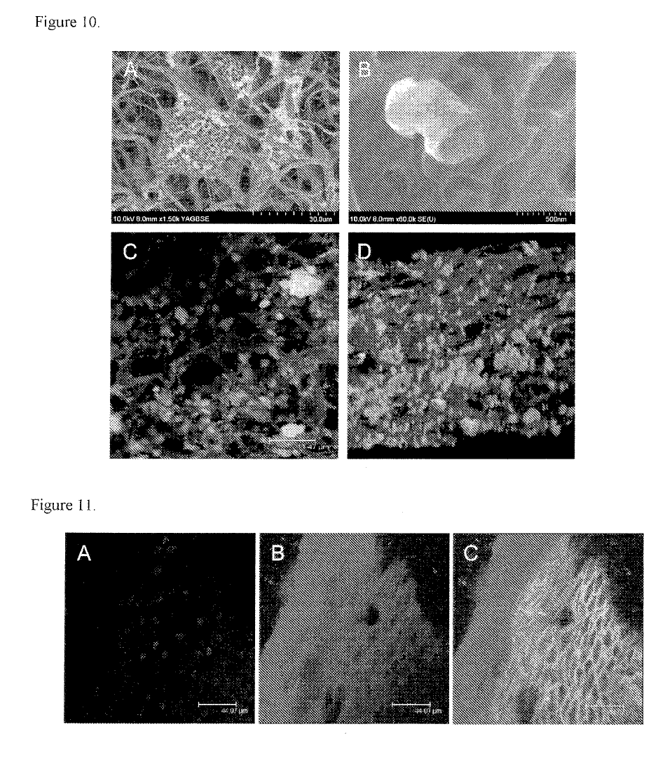

Fluorescent labelling of nanofibers: The nanofibrous layer prepared from PCL was labelled with the fluorescent dye lissamine-rhodamine. The nanofibrous layer was penetrated with labelled bacterial particles. Adsorption of bacterial particles on the nanofibrous layer was confirmed by the techniques of scanning electron microscopy and confocal microscopy (FIG. 10).

Preparation of the nanofibrous layer with microparticles of the “bacterial ghosts” type: The suspension of “bacterial ghosts” was prepared from 1 mg of BG lyophilisate in 1 ml of water using the tray-shaped ultrasound. The nanofibrous layer was immersed in this suspension and the vial was placed into the tray-shaped ultrasound for 5 minutes.

### Example 7

### Penetration of Nanoparticles (PLGA or Liposomes) Into the Sublingual and Buccal Porcine Mucosa After Their Release From the Nanofiber Mucoadhesive Particle Carrier

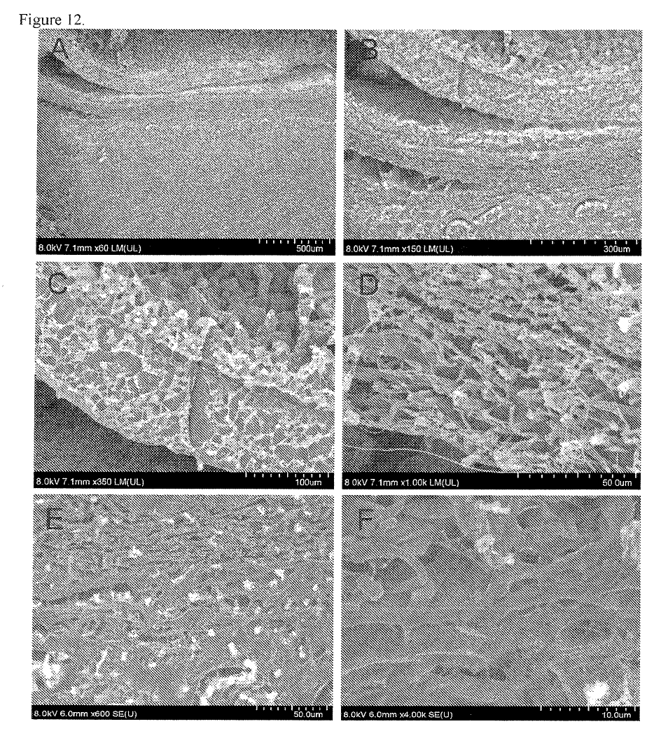

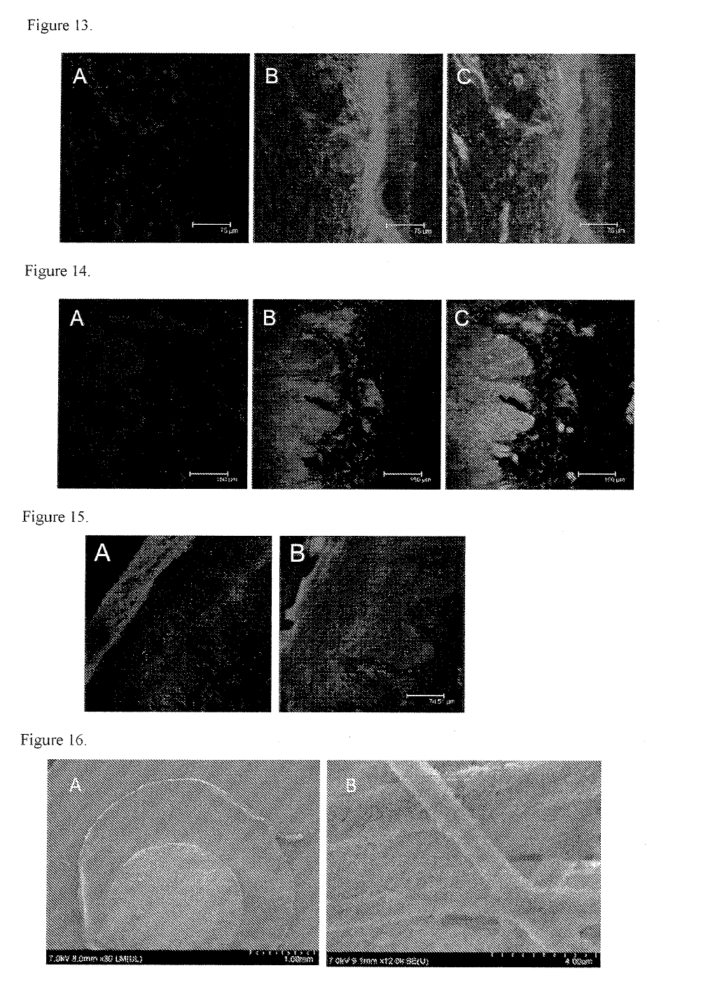

Penetration of nanoparticles from a nanofiber mucoadhesive carrier to the mucosa was confirmed in cross sections after incubation of the carrier adhered to freshly excised porcine sublingual and buccal mucosa (FIGS. 11, 12 and 13).

Preparation of the nanoparticle carrier: The nanoparticle carrier was prepared according to the procedure described in Example 1. The nanofibrous layer was impregnated with a solution of PLGA-PEG nanoparticles or PEG liposomes labelled with lissamine-rhodamine. To facilitate the penetration of nanoparticles into the mucosa, a suspension of PLGA-PEG nanoparticles in 1% sodium deoxycholate was used for impregnation of the nanofiber layer (FIGS. 14, 15).

Application of nanoparticles by means of a mucoadhesive system: A nanofiber mucoadhesive carrier with fluorescently labelled nanoparticles (PLGA-PEG or PEG liposomes dyed with lissamine-rhodamine) was administered to freshly excised sublingual mucosa by gentle pressure (see FIG. 21). Tissue samples were incubated at 37° C. for 4 hours. Then they were then quickly frozen in liquid nitrogen and stored at −75° C.

Preparation of tissue cross-sections: Cross-sections of 10-μm thickness were cut on a Cryo-cut instrument (Leica), fixed with acetone, and if needed, nuclei (blue, Sytox Blue) and actin (green, Alexa Fluor® 488 Phalloidin) were stained.

### Example 8

### Penetration of PLGA-PEG Nanoparticles Into Mouse Sublingual Mucosa

The sublingual mucosa contains different types of immune cells involved in immune response of the body and in inducing tolerance to the present antigens. Many types of particles (nanoparticles/microparticles) are suitable carriers of antigens. The particles allow us to combine antigens with immunomodulatory agents capable of influencing the resulting immune response.

The structure of sublingual mucosa differs between rodents, humans and pigs. It differs mainly in the degree of keratinization, which is a barrier to penetration of nanoparticles into the mucosa.

In an in vivo mouse model, no spontaneous penetration of PLGA nanoparticles into the sublingual mucosa was observed in contrast to porcine mucosa (see FIGS. 16 and 17). In in-vivo experiments, physiological functions of the immune system cells are not suppressed as it is in ex vivo experiments carried out on porcine mucous membranes (see FIGS. 11 to 15) and it is possible to observe particle the internalisation by phagocytic cells involved in the regulation of the immune response/tolerance.

In the experiment, the occurrence of large amounts of MHC II-positive cells capable of phagocytosis was confirmed in the mouse sublingual area where the mucoadhesive system for nanoparticles was administered (FIG. 17A). Phagocytosis of PLGA-PEG nanoparticles by specialized cells was also confirmed.

Preparation of PLGA-PEG nanoparticles: see Example, Embodiment 3

Administration of nanoparticles by means of the carrier into the mouse sublingual area: Adhesive system with a 4-mm diameter was slightly pressed against the mucosa in the sublingual area of a mouse. Administration time was 4 hours. After this time, the mouse was sacrificed and frozen in n-heptane of a temperature of −70° C.

Preparation of a cross-section of tissue: see Example, Embodiment 7

Evaluation of the experiment: Internalization of nanoparticles in specialized cells was localized by confocal microscopy.

### Example 9

### Lyophilisation of the Nanofibrous Layer With Impregnated Nanoparticles

If required by the nature of the nanoparticles and/or physiologically active substances carried by them, long-term stability of nanoparticles and/or the carried physiologically active substances which penetrated into the nanofibrous layer, can be achieved by lyophilisation or by simple drying.

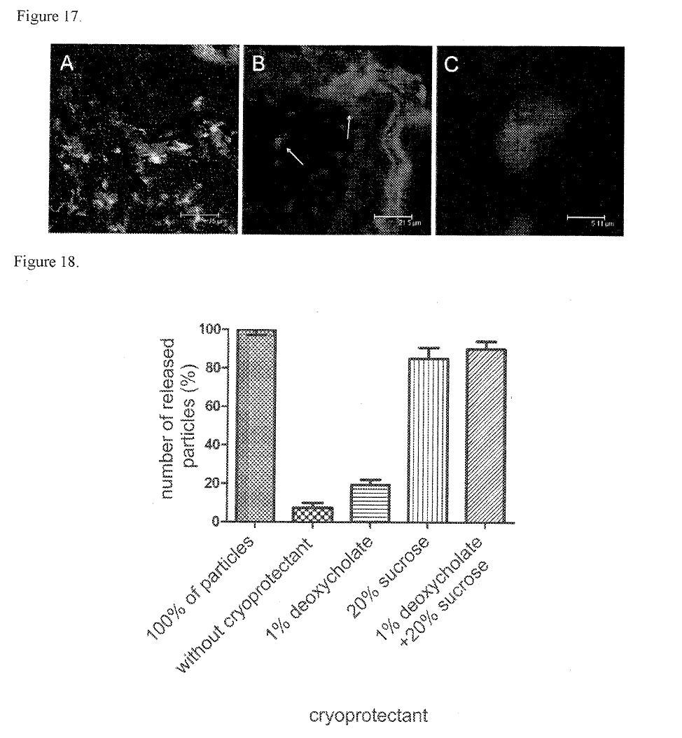

Depending on the nature of the nanoparticles, the amount of particles releasable from the nanofibrous layer can be considerably influenced by the addition of other substances to the solution of nanoparticles. The addition of cryopreservation agents (for example saccharides, such as sucrose, trehalose) and/or surfactants appear advantageous (FIG. 18).

Preparation of a PLGA-PEG nanoparticle suspension: PLGA-PEG nanoparticles (nanoparticle preparation see Example, Embodiment 3) were prepared as a suspension in water, 1% sodium deoxycholate, 20% sucrose or a mixture of 1% sodium deoxycholate and 20% sucrose.

Preparation of the nanofibrous layer: The amount of particles releasable from the matrix after lyophilisation was monitored in nanofibrous layers prepared from PCL.

Penetration of PLGA-PEG nanoparticles into the nanofiber matrix: Nanofiber matrix made from polycaprolactone, with the layer thickness of 15 μm and an area of 0.5 cm², was penetrated by PLGA-PEG nanoparticles by immersion into the prepared solution and sonication in an ultrasonic bath for 5 minutes.

Lyophilization of the nanofibrous layer with PLGA-PEG nanoparticles: After penetration, the samples were immediately frozen on dry ice so as to prevent drying of the solution. Frozen samples were lyophilized. The effect of cryoprotectants and surfactants was tested for the amount of releasable nanoparticles from the nanofibrous layer.

Release of PEG-PLGA nanoparticles from the nanofiber layer: Individual lyophilised nanofiber layers with nanoparticles were transferred into 500 μl of MilliQ -filtered water (20 nm Anotop filter, Millipore). The release of nanoparticles was carried out for 30 minutes while stirring on a shaker.

Determination of the number of released nanoparticles: Released amount and size of the PLGA-PEG nanoparticles was determined by “Nanoparticle Tracking Analysis” (NanoSight, Malvern, UK).

### Example 10

### Penetration of Nanoparticles (PLGA and Liposomes) Into the Sublingual Mucosa of a Piglet In Vivo After Their Administration, Using a Nanofiber Mucoadhesive Carrier

Preparation of the nanoparticle carrier: The nanoparticle carrier was prepared as described in Example, Embodiment 1. The nanofibrous layer was impregnated with a solution of PLGA-PEG nanoparticles or PEG liposomes labelled by lissamine-rhodamine. To facilitate penetration of nanoparticles into the mucosa, PLGA-PEG nanoparticle suspension in 1% sodium deoxycholate was used for impregnation of the nanofiber layer.

Administration of nanoparticles by the mucoadhesive system: A nanofiber mucoadhesive carrier with fluorescently labelled nanoparticles (PLGA-PEG rhodamine or PEG liposomes) was administered to the sublingual mucosa or buccal mucosa of a piglet (15 kg) applying a slight finger pressure. During the administration, the piglet was under general anaesthesia (injection of a short-acting anaesthetic). After two hours, the pig was again put into general anaesthesia and euthanized. Adjacent tissue with the particle carrier and a regional lymph node were excised and cross sections of tissues were prepared for the evaluation.

In-vivo penetration of particles: Penetration of nanoparticles from the nanofiber mucoadhesive carrier into the mucosa and regional lymph nodes was confirmed in cross-sections after oral mucosal administration of the carrier to the sublingual or buccal mucosa of the pig (FIG. 23).

1. A mucoadhesive (e.g. particle) carrier, characterized in that it comprises:
a nanoscaffold (or matrix) carrying or comprising at least one substance or API (e.g. in the form of particles), and
a mucoadhesive (layer),
wherein the mucoadhesive (layer), on at least a part of its surface, can adhere (to a mucosa) or overlap with the nanoscaffold.

2. The mucoadhesive carrier according to claim 1, characterized in that:
a) the nanoscaffold contains or has pores having the size of from 10 nm to 1,000 μm and/or is a nanofibrous layer of a thickness in the range 0.1 to 1,000 μm; or
b) comprises a layer of biocompatible polymers or a mixture thereof.

3. The mucoadhesive carrier according to claim 1, characterized in that:
a) the mucoadhesive layer (at least partially) overlaps the nanoscaffold, an edge of the mucoadhesive layer overlaps an edge of the nanoscaffold and/or the mucoadhesive layer surrounds the nanoscaffold along an edge; or
b) it is adapted for application onto a target mucosa, the nanoscaffold faces the mucosa (e.g. in the same direction as the mucoadhesive) and/or part of the mucoadhesive layer overlapping the nanoscaffold is adapted to adhesively fix (adhere) the mucoadhesive carrier to the target mucosa.

4. The mucoadhesive carrier according to claim 1, characterized in that it further comprises a cover layer (suitably not having mucoadhesive properties) which does not allow permeation of the substance;
optionally wherein the order of the layers is either nanoscaffold—mucoadhesive layer—cover layer,
or the nanoscaffold is adjacent to the cover layer (over at least part of its surface) and/or
the mucoadhesive layer is adjacent to the cover layer over at least part of its surface.

5. The mucoadhesive carrier according to claim 1, characterized in that it further comprises an intermediate layer (preferably not having mucoadhesive properties) which does not allow permeation of the substance therethrough,
preferably said intermediate layer being deposited or located between the nanoscaffold and the mucoadhesive and/or cover layer (to which it is may be attached).

6. The mucoadhesive carrier according to claim 4, characterized in that the cover layer and/or intermediate layer (e.g. water) are insoluble or are prolonged or sustained release layer(s).

7. The mucoadhesive carrier according to claim 1, characterized in that the particles are in the form of, or comprise, liposomes, nanoparticles, microparticles or macromolecules and are suitably anchored to, adsorbed or located in (the nanofibers of) the nanoscaffold.

8. The mucoadhesive carrier according to claim 7, additionally comprising at least component that is (at least one) excipient, preferably an absorption accelerator and/or excipients that may facilitate release of the particles (carried to the mucosal surface) and/or an excipient that may facilitate penetration of the particles through a mucin layer and/or an excipient that may facilitate penetration of the particles into (deeper) layers of the mucosa.

9. The mucoadhesive carrier according to claim 1, characterized in that the nanoscaffold comprises nanofibres, whose surface is preferably modified by physical or chemical treatment, or treatment with a chemical oxidizing agent, or a process which is plasma treatment, sodium hydroxide solution treatment, hydrophilic electroneutral polymer modification, adsorption of surfactants and/or influence or modification of the surface charge or the degree of particle wettability.

10. A mucoadhesive delivery system comprising:
a) a matrix comprising at least one active pharmaceutical ingredient (API); and
b) a mucoadhesive (or adhesive means) capable of adhering the system to a mucosa.

11. A system according to claim 10 wherein:
a) the matrix comprises a nanoscaffold, preferably comprising biocompatible polymer(s), and optionally having pores of from 10 nm to 1000 μm;
b) the mucoadhesive is a layer or adhesive portion and/or is capable of, or adapted to, secure, attach or adhere the system to a mucosa;
c) the mucoadhesive layer (at least in one part thereof) overlaps the matrix, so as to expose part of the layer to the mucosa; and/or
d) the API is in the form of particles.

12. A method for preparing a mucoadhesive carrier according to claim 1, characterized in that a nanoscaffold is joined or contacted with a mucoadhesive layer and/or a cover layer, and preferably an intermediate layer is inserted between said layers (usually before joining the layers).

13. The method according to claim 12, characterized in that the mucoadhesive layer and/or the intermediate layer and/or the cover layer is formed first, preferably by an electrostatic spinning method (e.g. in the form of nanofibres) or by (polymer) spraying, and optionally then the layers are firmly joined in the desired order, and preferably the nanoscaffold is prepared in situ onto the mucoadhesive layer and/or the cover layer and/or the intermediate layer.

14. The method according to claim 12, characterized in that a substance and optionally at least one excipient is deposited onto the nanoscaffold, either after its preparation or after joining all layers of the mucoadhesive carrier, preferably said substance and/or excipient being in the form of a solution, colloid or suspension.

15. The method according to claim 14, characterized in that the mucoadhesive carrier with the substance is then lyophilized.

16. The mucoadhesive carrier according to claim 1, characterized in that it is adapted for administration manually or by a delivery device (suitably by pressure directly to the target mucosa) preferably so that the nanoscaffold faces or adheres to the mucosa.

17. The mucoadhesive carrier according to claim 1 for:
a) use as or with a vaccine, preferably for delivery to mucosal surface(s), especially for sublingual vaccination and/or immunotherapy; or
b) delivery of therapeutic particle(s) preferably with local and/or systemic effect.

18. A mucoadhesive carrier according to claim 1 for use in medicine and as, or in, a medicament.

19. A mucoadhesive carrier according to claim 1 for use in a method of treatment and/or diagnosis of the human or animal body.

20. A mucoadhesive carrier according to claim 1 in the manufacture of a medicament or vaccine for the prophylaxis treatment of a disease or condition in a human or animal.

21. A mucoadhesive carrier or system substantially as herein described with reference to the Examples and/or Figures/drawings.
